(19) United States
(12) Patent Application Publication
(10) Pub. No.: US 2023/0371834 A1
(43) Pub. Date: Nov. 23, 2023

(54) REUSABLE AIR CHARGER AND ASSOCIATED MULTI-LUMEN DISPOSABLE CATHETER, AND RELATED METHODS

(71) Applicant: Laborie Medical Technologies Corp., Portsmouth, NH (US)

(72) Inventors: Albert Meek, Enschede (NL); Ryan Bernier, Manchester, NH (US); Julian Peters, Newmarket, NH (US); Andrê Tremblay, Oka (CA); Calvin Turland, Verdun (CA); Jessica Dakkak, Saint-Laurent (CA); Louis-Simon Guay, Montreal (CA); Benoit Thibauit, Coteau-du-Lac (CA)

(21) Appl. No.: 18/318,971

(22) Filed: May 17, 2023

Related U.S. Application Data
(60) Provisional application No. 63/365,019, filed on May 19, 2022.

(51) Int. Cl. A61B 5/03; A61M 25/00; A61B 5/00; A61M 25/10

(52) U.S. Cl. CPC A61B 5/036; A61M 25/0026; A61B 5/6852; A61M 25/10181; A61M 25/1011; A61M 2025/0002; A61B 2562/168

(57) ABSTRACT

A diagnostic manometry device comprises a catheter including a plurality of lumens, a fluid charger configured to operatively couple with the catheter, and a charging mechanism for moving fluid simultaneously from fluid pressure chambers of the plurality of fluid pressure chambers into the respective lumens of the plurality of lumens when the catheter is operatively coupled with the fluid charger. Each fluid pressure chamber of the plurality of fluid pressure chambers is configured to be in fluid communication with a respective lumen of the plurality of lumens when the catheter is operatively coupled with the fluid charger. Each pressure sensor of the plurality of pressure sensors is located and configured to measure a fluid pressure change in a respective fluid pressure chamber of the plurality of fluid pressure chambers. Additional manometry devices and methods of forming a manometry device are also disclosed.

### CROSS-REFERENCE TO RELATED APPLICATION

This application claims the benefit under 35 U.S.C. § 119(e) of U.S. Provisional Patent Application Ser. No. 63/365,019, filed May 19, 2022, the disclosure of which is hereby incorporated herein in its entirety by this reference.

### TECHNICAL FIELD

This disclosure relates generally to a diagnostic manometry device that includes a reusable air charger and an associated multi-lumen disposable catheter, and to related methods of making and using such diagnostic manometry devices. In particular, embodiments of the present disclosure relate to such manometry devices wherein the air charger is configured to charge a plurality of lumens in the catheter with fluid simultaneously in the performance of a diagnostic procedure, and to related methods.

### BACKGROUND

Medical professionals often collect data and perform diagnostics relative to internal regions of the body of a patient. Gastroenterologists often use catheters and hand-held devices for the data collection and diagnostics relative to the gastrointestinal (GI) tract.

Anorectal Manometry (ARM) is a test often performed by GI specialists to analyze voluntary and involuntary contractions and/or relaxations of the anal rectum. Multiple tests are known to be performed as part of anorectal manometry, such as the balloon expulsion test of a rectal exam and many others. The use of the catheter and hand-held devices (e.g., FOB) in performing these tests has contributed to an increasing amount of data in the GI and ARM fields of study. Many of the tests and studies are not sufficiently standardized.

Furthermore, within ARM studies, there are different individualized procedures and associated devices, each involving complex routines, varied advantages, and unavoidable disadvantages. For example, non-high resolution ARM uses relatively few sensors and thus fewer measurements at wider intervals, which may present challenges to data interpretation. High Resolution Anorectal Manometry (HRAM) involves an increased number of sensors relative to non-high resolution ARM. High definition Anorectal Manometry (HDAM) may use hundreds (e.g., about 250) of sensors distributed radially and longitudinally along a catheter. Although HDAM offers increased data sensitivity, it is even more expensive than HRAM and often involves use of catheters of higher rigidity and larger diameter, which may cause elevated intra-anal pressure, especially in children.

Generally, each of the ARM procedures involves an initialization process to calibrate and prime the sensors, catheters, and hand-held devices. The initialization processes may be tedious, cumbersome, expensive, overcomplicated, and overwhelming to the medical professionals and assistants performing the initializations and calibrations, especially when they are performed multiple times throughout a day.

### BRIEF SUMMARY

In some embodiments, the present disclosure includes a fluid charger for use together with a multi-lumen catheter in a diagnostic manometry device. The fluid charger includes a housing, a releasable catheter connection mechanism configured to couple with a proximal end of a catheter including lumens defined within a body of the catheter, fluid pressure chambers, and pressure sensors for measuring fluid pressure changes in the fluid pressure chambers. Each fluid pressure chamber is in fluid communication with a respective fluid conduit leading to the catheter connection mechanism so as to fluidly couple each fluid pressure chamber with a respective lumen of a catheter when the catheter is operatively connected to the fluid charger. The fluid charger further includes a charging mechanism for moving fluid simultaneously from the fluid pressure chambers through the fluid conduits toward the catheter connection mechanism.

In additional embodiments, the present disclosure includes a diagnostic manometry device that includes a fluid charger as described herein, and a catheter comprising a plurality of lumens therein. The fluid charger may include a plurality of fluid pressure chambers, each fluid pressure chamber of the plurality of fluid pressure chambers configured to be in fluid communication with a respective lumen of the plurality of lumens when the catheter is operatively coupled with the fluid charger. The fluid charger further includes a plurality of pressure sensors, each pressure sensor of the plurality of pressure sensors located and configured to measure a fluid pressure change in a respective fluid pressure chamber of the plurality of fluid pressure chambers. The fluid charger also comprises a charging mechanism for moving fluid simultaneously from the fluid pressure chambers of the plurality of fluid pressure chambers into the respective lumens of the plurality of lumens when the catheter is operatively coupled with the fluid charger.

In yet further embodiments, the present disclosure includes a method of performing a diagnostic manometry procedure on a patient. The method comprises coupling a catheter including a plurality of lumens to a fluid charger as described herein. For example, the fluid charger may include a plurality of fluid pressure chambers, each of which may be in fluid communication with a respective lumen of the plurality of lumens upon the coupling of the catheter to the fluid charger. The fluid charger may include a plurality of pressure sensors, each pressure sensor of the plurality of pressure sensors located and configured to measure a fluid pressure change in a respective fluid pressure chamber of the plurality of fluid pressure chambers. The fluid charger may further include a charging mechanism for moving fluid simultaneously from the fluid pressure chambers of the plurality of fluid pressure chambers into the respective lumens of the plurality of lumens. The method further comprises inserting the catheter into a body of patient, and manipulating the charging mechanism so as to simultaneously move fluid from the fluid pressure chambers of the plurality of fluid pressure chambers into the respective lumens of the plurality of lumens. Changes in pressure in the fluid pressure chambers of the plurality of fluid pressure chambers are then sensed using the pressure sensors of the plurality of pressure sensors.

### BRIEF DESCRIPTION OF THE DRAWINGS

For a detailed understanding of the present disclosure, reference should be made to the following detailed description, taken in conjunction with the accompanying drawings, in which like elements have generally been designated with like numerals, and wherein:

FIG. 1 is a perspective view of a diagnostic manometry device including a multi-lumen catheter and associated fluid charger in accordance with embodiments of the present disclosure;

FIG. 2 is a cross-sectional view of the multi-lumen catheter of FIG. 1;

FIG. 3A is a cross-sectional view of certain components of the diagnostic manometry device of FIG. 1;

FIG. 3B is an enlarged portion of FIG. 3A;

FIG. 4 is a partial view of a rotatable disc and a mechanically associated fluid chamber plate, which form components of a charging mechanism of the fluid charger of FIG. 1;

FIG. 5 illustrates various internal components of the fluid charger of the device of FIG. 1;

FIG. 6 illustrates a lower housing and an internal printed circuit board of the fluid charger of the device of FIG. 1;

FIG. 7 illustrates a catheter connector on the proximal end of the catheter of the device of FIG. 1, disconnected from the fluid charger;

FIG. 8 illustrates the catheter connector of the catheter proximate to but disconnected from the release mechanism and the piston plate of the fluid charger of the device of FIG. 1;

FIG. 9 illustrates the catheter connector of FIG. 8 fully coupled to the release mechanism and the piston plate of the fluid charger;

FIG. 10 illustrates a proximal end of the catheter connector of the catheter of the device of FIG. 1;

FIG. 11 is cross-sectional view of the catheter connector and distal end of the catheter of the device of FIG. 1;

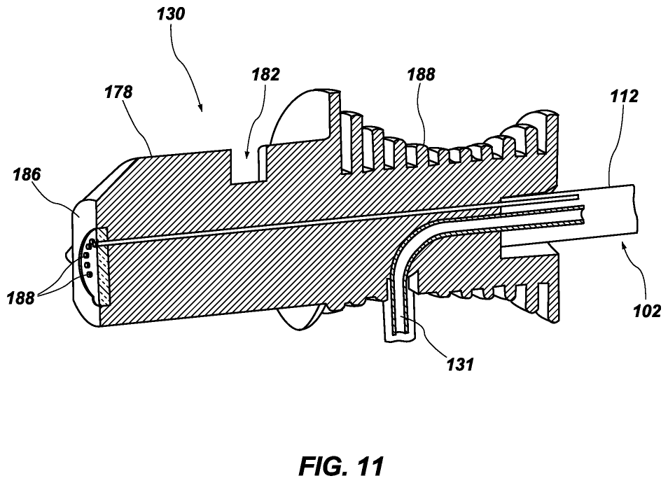

FIG. 12 is a partial cross-sectional view of the catheter connector and the piston plate of the fluid charger of the device of FIG. 1 illustrating fluid flow paths therethrough;

FIG. 13 is a schematic diagram of a diagnostic manometry device in accordance with embodiments of the present disclosure, including one such as that illustrated in FIG. 1; and

FIG. 14 is schematic diagram of a diagnostic manometry device in accordance with additional embodiments of the present disclosure, including one such as that illustrated in FIG. 1.

### DETAILED DESCRIPTION

The illustrations presented herein are not actual views of any particular diagnostic manometry device, or any component thereof, but are merely idealized representations employed to describe example embodiments of the present disclosure. The following description provides specific details of embodiments of the present disclosure in order to provide a thorough description thereof. However, a person of ordinary skill in the art will understand that the embodiments of the disclosure may be practiced without employing many such specific details. The drawings accompanying the application are for illustrative purposes only, and are not drawn to scale.

As used herein, the singular forms following “a,” “an,” and “the” are intended to include the plural forms as well, unless the context clearly indicates otherwise.

As used herein, the term “may” with respect to a material, structure, feature, method, or act indicates that such is contemplated for use in implementation of an embodiment of the disclosure, and such term is used in preference to the more restrictive term “is” so as to avoid any implication that other compatible materials, structures, features, and methods usable in combination therewith should or must be excluded.

As used herein, any relational term, such as “first,” “second,” “top,” “bottom,” “upper,” “lower,” “above,” “beneath,” “side,” “upward,” “downward,” etc., is used for clarity and convenience in understanding the disclosure and accompanying drawings, and does not connote or depend on any specific preference or order, except where the context clearly indicates otherwise. For example, these terms may refer to an orientation of elements of any cutting element when utilized in a conventional manner. Furthermore, these terms may refer to an orientation of elements of any cutting element as illustrated in the drawings.

As used herein, the term “substantially” in reference to a given parameter, property, or condition means and includes to a degree that one skilled in the art would understand that the given parameter, property, or condition is met with a small degree of variance, such as within acceptable manufacturing and/or measurement tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “about” used in reference to a given parameter is inclusive of the stated value and has the meaning dictated by the context (e.g., it includes the degree of error associated with measurement of the given parameter, as well as variations resulting from manufacturing tolerances, etc.).

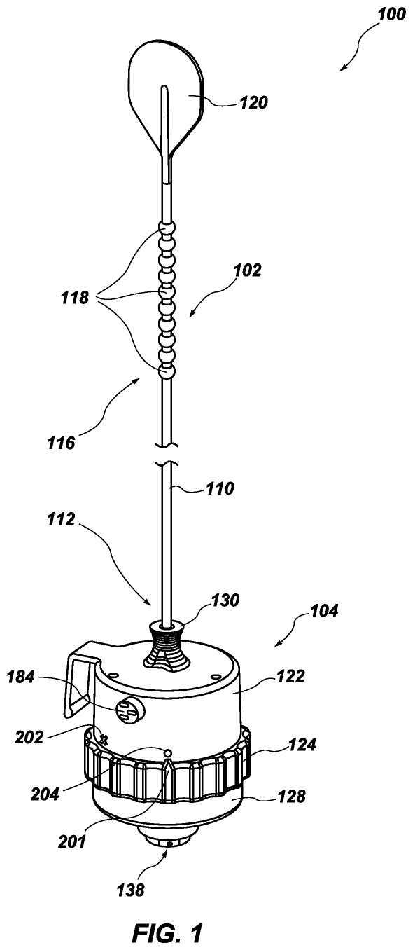

FIG. 1 illustrates a diagnostic manometry device 100 according to an embodiment of the present disclosure. The diagnostic manometry device 100 includes a catheter 102 and a fluid charger 104. The catheter 102 is configured to be inserted into the body of a patient and used to measure changes in pressure applied to exterior surfaces of the catheter 102 while the catheter 102 is disposed within the body. In particular, the diagnostic manometry device 100 may be used in anorectal manometry procedures, such as in High Resolution Anorectal Manometry (HRAM) procedures. In such procedures, the catheter 102 may be inserted into the body of the patient through the anus, and the diagnostic manometry device 100 may be used to measure pressure generated by the anal sphincter and/or colon for the evaluation of anal incompetence and fecal incontinence, for example. Of course, diagnostic manometry devices as described herein may also be used in other manometry diagnostic procedures, such as in esophageal manometry procedures, for example.

The catheter 102 of the diagnostic manometry device 100 is configured to be operably connected with the fluid charger 104. The catheter 102 may be disposable, while the fluid charger 104 is reusable (e.g., non-disposable). In some embodiments, the fluid charger 104 may include a handle to facilitate manual manipulation thereof during use.

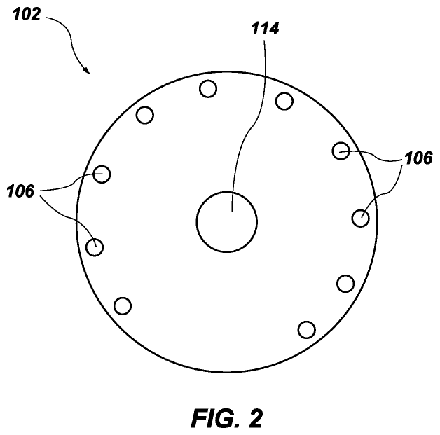

The catheter 102 has a plurality of lumens 106 (FIG. 2) extending longitudinally therethrough from a proximal end 112 of the catheter toward a distal end 116 of the catheter 102 and configured for fluid to pass therethrough. Each lumen 106 extends to and is in fluid communication with a respective pressure transmission chamber 118 of a plurality of pressure transmission chambers 118. The body of the catheter 102 may comprise an elastomeric material such as, for example, silicone, and portions of the body of the catheter 102 that define the pressure transmission chambers 118 may be slightly expandable, such that the pressure transmission chambers 118 can be deflated and collapsed during insertion of the catheter 102 into the body of the patient. Once inserted, fluid (e.g., a gas, such as air, for example) may be caused to flow through the lumens 106 of the catheter 102 and into the pressure transmission chambers 118 so as to inflate and expand, or “charge” the pressure transmission chambers 118 with fluid in preparation for the acquisition of manometry measurements. The pressure transmission chambers 118 may be positioned at or adjacent to the distal end 116 of the catheter 102.

The catheter 102 may further include an additional lumen 114 (FIG. 2) extending longitudinally through the catheter to, and in fluid communication with, an inflatable balloon 120 at the distal end 116 of the catheter. The inflatable balloon 120 may be inflated during a manometry diagnostic procedure by causing fluid to flow through the additional lumen 114 to the inflatable balloon 120 so as to induce sensations (and to measure responsive pressure changes adjacent the catheter 102) in the patient through the expansion of the balloon 120 with the fluid.

As noted above, the catheter 102 may include the plurality of lumens 106 and associated pressure transmission chambers 118. By way of example, the catheter 102 may include at least four lumens 106 and respectively associated pressure transmission chambers 118. In additional embodiments, the catheter 102 may include at least ten lumens 106 and respectively associated pressure transmission chambers 118. Of course, catheters having more or less lumens 106 and associated fluid pressure chambers are also contemplated and may be employed in embodiments of diagnostic manometry devices of the present disclosure.

Each pressure transmission chamber 118 may also be characterized as an expandable cylindrical balloon carried concentrically along the catheter 102, although they are relatively smaller than the balloon 120 intended for stimulation located at the distal end 116 of the catheter 102. In other words, each of the plurality of pressure transmission chambers 118 may comprise an interior volume contained within an expandable balloon, the interior sidewalls of the expandable balloon defining the pressure transmission chamber therein.

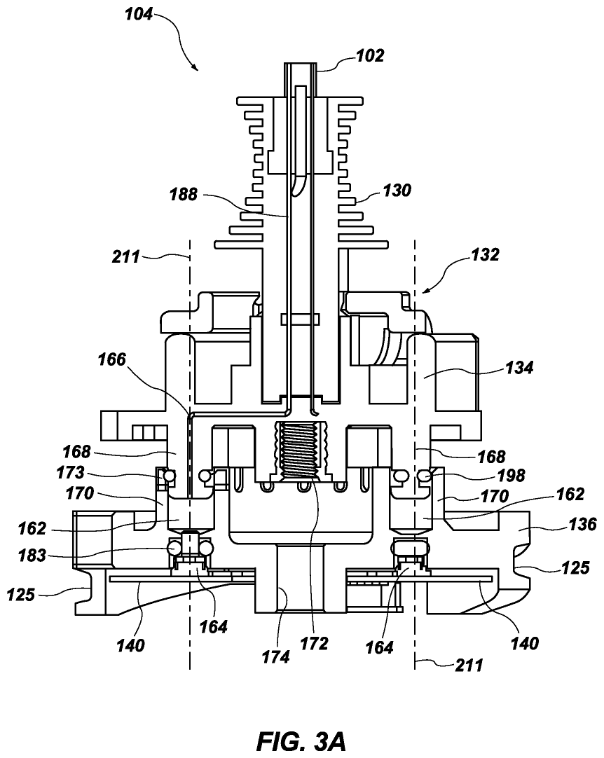

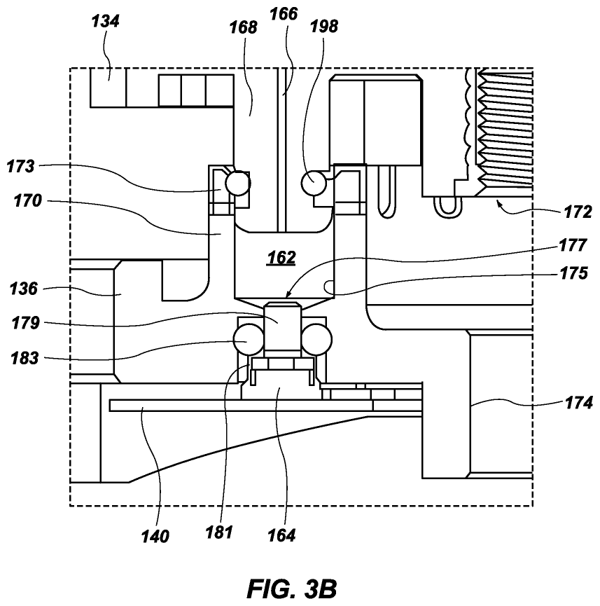

The catheter 102 and the fluid charger 104 are operatively coupled together, and the fluid charger 104 includes pressure sensors 164 (FIGS. 3A and 3B) that may be used to detect pressure and/or fluid pressure changes within the pressure transmission chambers 118 and associated lumens 106.

With continued reference to FIG. 1, the fluid charger 104 includes an upper housing 122, a moveable component in the form of a rotatable disc 124, and a lower housing 128. The proximal end 112 of the catheter 102 is coupled to a relatively rigid catheter connector 130, and the catheter connector 130 can be releasably connected and operatively coupled with the upper housing 122 using a snap fit connection, for example. A depressible button 184 on the upper housing 122 may be used to selectively decouple the catheter connector 130 from the upper housing 122 so as to detach the catheter 102 from the fluid charger 104.

An electrical connector 138 may be carried by the lower housing 128 and includes multiple pins used to electrically couple the pressure sensors and associated electronic components of the fluid charger 104 with an external control unit (e.g., a computer, laptop, or a dedicated electronic controller). In some embodiments, the external control unit may handle the bulk of the digital signal processing requirement for obtaining the desired end results (e.g., graphical representations of the acquired data, conclusions, treatment recommendations, etc.) to be obtained by the overall diagnostic system.

Referring to FIG. 2, as noted above, the catheter body 110 includes the plurality of lumens 106 and the additional lumen 114 defined within the interior of the catheter body 110. The lumens 106 extend longitudinally between the proximal end 112 and the pressure transmission chambers 118 near, such as at or adjacent to, the distal end 116 of the catheter 102. The additional lumen 114 extends longitudinally between the proximal end 112 of the catheter 102 and the balloon 120 at the distal end 116 of the catheter 102. The additional lumen 114 is not in fluid communication with any of the other lumens 106 within the catheter, and is not in fluid or electrical communication with the fluid charger 104 during use of the manometry device 100.

The catheter body 110 comprises a flexible, fluid impermeable material, such as silicone, thereby creating a barrier between bodily fluids of a patient and fluid (e.g., gas or air) used to charge or inflate the pressure transmission chambers 118 and/or the balloon 120. For example, the catheter body 110 may not include any direct fluid path between the exterior of the catheter 102 and the interior regions of the catheter 102, such as within an interior of the plurality of pressure transmission chambers 118 and associated lumens 106 or within an interior of the balloon 120 and associated additional lumen 114. In some embodiments, the catheter body 110 may be formed by extrusion, injection molding, or another similar process.

FIG. 3A illustrates the catheter connector 130 coupled to certain components of the fluid charger 104. The upper housing 122, rotatable disc 124, and lower housing 128 (FIG. 1) are not shown in FIG. 3A to reveal the illustrated internal components for purposes of illustration. FIG. 3B is an enlarged view of a portion of FIG. 3A.

Referring collectively to FIGS. 3A and 3B, the fluid charger 104 includes a plurality of fluid pressure chambers 162, and each fluid pressure chamber 162 is configured to be in fluid communication with a respective lumen 106 of the catheter 102 and associated pressure transmission chamber 118 when the catheter 102 is operatively coupled with the fluid charger 104. The fluid charger 104 further includes a plurality of pressure sensors 164. The pressure sensors 164 are mounted on a sensor printed circuit board (PCB) 140. Each pressure sensor 164 is located and configured to measure a fluid pressure change in a respective fluid pressure chamber 162 in the fluid charger 104, and hence, in the associated lumen 106 and pressure transmission chamber 118 in the catheter 102. Furthermore, the fluid charger 104 further includes a charging mechanism for moving fluid simultaneously from the fluid pressure chambers in the fluid charger 104 into the respective lumens 106 and associated pressure transmission chambers of the catheter 102 when the catheter 102 is operatively coupled with the fluid charger 104, as described in further detail below.

The fluid charger 104 includes a plurality of pistons 168 and a plurality of piston chambers 170. Each piston chamber 170 is sized and configured to receive a respective piston 168 of the plurality therein in a fluid-tight manner. Each piston 168 and respective piston chamber 170 define a respective fluid pressure chamber 162 of the plurality therebetween.

The pistons 168 are carried on a piston plate 134. The pistons 168 may be integral parts of the piston plate 134. The piston plate 134 is in the form of a generally circular disc, with the pistons 168 formed in a circular array on a common side of the piston plate 134. The pistons 168 extend generally parallel to one another along longitudinal axes 211 thereof. The piston plate 134 is fixed in position relative to the upper housing 122 (FIG. 1) within the fluid charger 104.

The piston chambers 170 are carried on a piston chamber plate 136. The piston chambers 170 may be integral parts of the piston chamber plate 136. The piston chamber plate 136 is also in the form of a generally circular disc, with the piston chambers 170 formed on a common side of the piston chamber plate 136. The piston chambers 170 extend generally parallel to one another along the longitudinal axes 211 thereof. The piston chamber plate 136 is configured to move relative to the piston plate 134 and the upper housing 122 (FIG. 1) within the fluid charger 104 in the longitudinal direction parallel to the axes 211, as described in further detail below. The sensor PCB 140 is mounted to the underside of the piston chamber plate 136 opposite the piston chambers 170. By employing board-mounted pressure sensors 164, forces used to fix the sensor PCB 140 to the piston chamber plate 136 also facilitate sealing the respective pressure sensors 164 to the piston chambers 170 within the respective fluid pressure chambers 162.

Each piston 168 extends into and is at least partially disposed within a piston chamber 170 so as to define a fluid pressure chamber 162 between the piston 168 and associated piston chamber 170, as shown in FIGS. 3A and 3B. Thus, during operation, interior sidewalls of the piston chamber plate 136 that define the piston chambers 170 are disposed laterally adjacent sidewalls of the respective pistons 168. Each piston chamber 170 of the fluid charger 104 is sized, aligned, and configured to receive a respective piston 168 therein in a fluid-tight manner. For example, an inner diameter of a piston chamber 170 may be substantially equivalent to an outer diameter of an associated, respective piston 168. The end of each piston 168 may have a beveled or chamfered perimeter to ensure smooth movement relative to the respective piston chamber 170.

Each fluid pressure chamber 162 is thus in fluid communication with a respective lumen 106 and pressure transmission chamber 118 of the catheter 102 when the catheter 102 is operatively connected to the fluid charger 104. In particular, an internal fluid conduit 166 extends from each fluid pressure chamber 162 to a respective lumen 106 of the catheter 102 at the proximal end 112 of the catheter 102 when the catheter 102 is operatively connected to the fluid charger 104 by way of the catheter connector 130. In some embodiments, the catheter connector 130 may include rigid tubes 188 extending therethrough that fluidly couple the lumens 106 to respective internal fluid conduits 166.

As shown in FIG. 3B, the sensor PCB 140 may be mounted to the piston chamber plate 136 such that each pressure sensor 164 is located and configured to measure pressure within a respective fluid pressure chamber 162. For example, converging sloped sidewalls 175 and an opening 177 may be formed in the piston chamber plate 136 within each piston chamber 170 at an end thereof opposite the respective piston 168 so as to define a fluid transmission column 179 in the opening 177. The pressure sensor 164 may be located adjacent the fluid transmission column 179 so as to detect and measure the pressure in the fluid transmission column 179 and, hence, within the pressure chamber 162 and the respective lumen 106 and pressure transmission chamber 118 of the catheter 102. An O-ring 183 may be used to ensure a fluid tight seal between the pressure sensor 164 and/or sensor PCB 140 and the piston chamber plate 136.

A respective pressure sensor 164 may be positioned relative to the converging sloped sidewall 175, which directs the fluid into an opening 177 of a fluid transmission column 179. The fluid transmission column 179 may be in fluid communication with the pressure sensor 164. As a non-limiting example, each pressure sensor 164 may comprise a digital pressure sensor. Each pressure sensor 164 may comprise a small piezoresistive pressure sensor providing a digital output for reading pressure over the anticipated pressure ranges at anticipated operating temperature ranges. Each pressure sensor 164 may be calibrated and compensated over a specific temperature range for sensor offset, sensitivity, temperature effects, and non-linearity using an on-board Application Specific Integrated Circuit (ASIC). As a non-limiting example, each pressure sensor 164 may comprise a MICROPRESSURE MPR SERIES sensor, which are manufactured and sold by Honeywell International Inc. of Charlotte, North Carolina.

Each pressure sensor 164 may comprise one or more calibrated strain gauges that correlate a detected level of strain applied to a component of the pressure sensor 164 (e.g., a piezoelectric member) that is correlated to a pressure applied to the strained component. The detected strain may be output as one or more digital output signals.

In other alternative embodiments, the fluid transmission column 179 may be a moveable piston adjacent a semispherical sidewall 181 such that pressure is transmitted from the fluid transmission column 179 to the circumferential semispherical sidewall 181, and then to annular shaped strain gauges that correspond to the bottom circumferential surface of the semispherical sidewall 181. An O-ring 183 prevents the fluid within the fluid transmission column 179 from exiting other than through the opening 177.

In additional embodiments, the plurality of pressure sensors 164 incorporate an alternative method of measuring circumferential pressure, such as is discussed in European Patent No. 2,417,906 B1, titled METHOD OF CONFIGURING A PRESSURE SENSING CATHETER, AND CATHETER SHEATH, by Thomas R. Parks, the disclosure of which is incorporated by this reference in its entirety.

The fluid pressure chambers 162 and fluid conduits 166 in the fluid charger 104 are capable of simultaneous pressurization and/or simultaneous fluid evacuation as described in further detail below.

In the embodiment illustrated in the figures, the piston plate 134 is fixed in position relative to the upper housing 122 (FIG. 1) within the fluid charger 104, and it is the piston chamber plate 136 that is movable relative to the piston plate 134 and the upper housing 122 (FIG. 1) within the fluid charger 104. In additional embodiments, however, the configuration could be reversed. In other words, the piston chamber plate 136 could take the place of the piston plate 134 and be fixed in position relative to the upper housing 122 (FIG. 1) within the fluid charger 104, and the piston plate 134 could take the place of the piston chamber plate 136 and be movable relative to the piston chamber plate 136 and the upper housing 122 (FIG. 1) within the fluid charger 104.

Thus, the fluid charger 104 is configured for relative movement between the pistons 168 and the piston chambers 170 with one of either the pistons 168 or the piston chambers 170 is rigidly connected to the upper housing 122 and lower housing 128 of the fluid charger 104, and the other of either the pistons 168 or the piston chambers 170 is movable relative to the upper housing 122 and lower housing 128 of the fluid charger 104. This enables a user to provide relative movement between the pistons 168 and piston chambers 170, which causes the volume of fluid within the fluid pressure chambers 162 to change (increase or decrease, depending on the direction of movement). In other words, the piston plate 134 or the piston chamber plate 136 is moveable, while the other is rigidly connected to upper housing 122 and lower housing 128 to facilitate the simultaneous charging of the pressure transmission chambers 118 in the catheter 102 with fluid.

More specifically, to charge the pressure transmission chambers 118 in the catheter 102 with fluid, the fluid charger 104 includes a charging mechanism for moving the piston chamber plate 136 toward the piston plate 134, which causes the volumes of the pressure chambers 162 to decrease. This decrease in the volume of the pressure chamber 162 causes displacement of fluid within the pressure chambers 162 into the fluid conduits 166, and into the respectively associated lumens 106 and pressure transmission chambers 118 of the catheter 102. This displacement of fluid from the pressure chambers 162 in the fluid charger 104 and into the pressure transmission chambers 118 of the catheter 102 occurs simultaneously and substantially uniformly. This provides an advantage in that each of the lumens 106 and pressure transmission chambers 118 of the catheter 102 may be charged with fluid simultaneously rather than sequentially, which requires less time, and more uniformly relative to known devices and methods.

Thus, the charging mechanism of the fluid charger 104 is configured to enable relative movement between the plurality of pistons 168 and the plurality of piston chambers 170 in unison so as to simultaneously and substantially uniformly change volumes of the fluid pressure chambers 162 of the plurality defined between the respective pistons 168 and piston chambers 170. More specifically, the charging mechanism may comprise a moveable component moveable between a first position and a second position, movement of the moveable component from the first position to the second position causing the relative movement between the plurality of pistons 168 and the plurality of piston chambers 170.

Referring again briefly to FIG. 1, the charging mechanism of the fluid charger 104 comprises a moveable component, herein the form of a rotatable disc 124, that is moveable between a first position and a second position. Movement of the moveable component from the first position to the second position causing the relative movement between the plurality of pistons 168 and the plurality of piston chambers 170. In this embodiment, the moveable component is a rotatable disc 124 that is exposed on the exterior of the fluid charger 104 that can be rotated by a user between a first rotational position and a second rotational position. The rotatable disc 124 may comprise grooves, ridges, or raised surfaces to facilitate manual gripping of the rotatable disc 124 by the user.

The charging mechanism further comprises a base member, and one of the plurality of pistons 168 and the plurality of piston chambers 170 being rigidly connected to the base member. In this embodiment, the base member is in the form of a plate. More particularly, the base member comprises the piston chamber plate 136, although in other embodiments it could be the piston plate 134 or any other plate or member to which either the pistons 168 or the piston chambers 170 are fixedly attached.

The moveable component of the charging mechanism (e.g., the rotatable disc 124) is operatively coupled with the base member of the charging mechanism (e.g., the piston chamber plate 136) such that movement of the moveable component from the first position to the second position causes the base member to move in a direction parallel to longitudinal axes 211 of the pistons 168 and respective piston chambers 170.

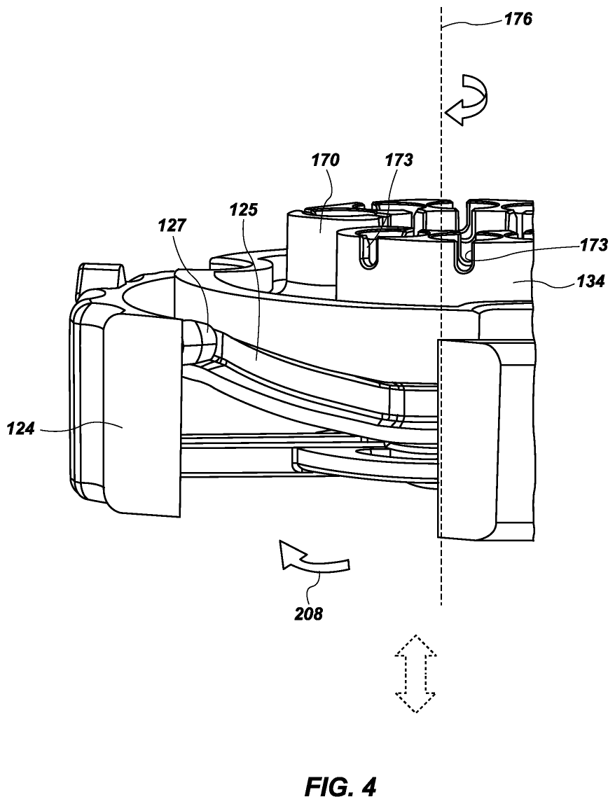

FIG. 4 illustrates a portion of the rotatable disc 124 and the mechanically associated piston chamber plate 136. The rotatable disc 124 is rotatable about a rotational axis 176 in either the clockwise or counterclockwise directions. As shown in FIG. 4, the radially outer surface of the piston chamber plate 136 and a radially inner surface of the rotatable disc 124 include mechanically cooperating features, at least one of which has a helical configuration such that relatively rotational movement between the rotatable disc 124 and the piston chamber plate 136 also results in relative translational movement between the rotatable disc 124 and the piston chamber plate 136. As a non-limiting example, a helically extending groove 125 may be formed on the radially outer surface of the piston chamber plate 136, and one or more protrusions 127 may be formed on the radially inner surface of the rotatable disc 124. The protrusions 127 on the rotatable disc 124 extend into the helical groove 125 on the piston chamber plate 136. The rotatable disc 124 cannot rotate about the rotational axis 176 due to the fact that the piston plate 134 (FIGS. 3A and 3B) is fixed in position relative to the upper housing 122 and the pistons 168 are respectively disposed in the piston chambers 170. The piston chamber plate 136 can translate linearly along the rotational axis 176, however. Thus, as the rotatable disc 124 is rotated by a user, the protrusions 127 on the rotatable disc 124 will be forced to translate along and through the groove 125 on the piston chamber plate 136, and the helical configuration of the groove 125 will cause the piston chamber plate 136 to move in the direction parallel to the rotational axis 176. In the configuration shown in FIG. 4, rotation of the rotatable disc 124 in the rotational direction shown by directional arrow 208 will cause the piston chamber plate 136 to move in the downward direction (from the perspective of FIG. 4), which will result in expansion of the volumes of the pressure chambers 162 and withdrawal of fluid from the pressure transmission chambers 118 and lumens 106 of the catheter 102. Rotation of the rotatable disc 124 in the opposite rotational direction (opposite to directional arrow 208) will cause the piston chamber plate 136 to move in the upward direction (from the perspective of FIG. 4), which will result in reduction of the volumes of the pressure chambers 162 and charging of fluid into the lumens 106 and the pressure transmission chambers 118 of the catheter 102.

Thus, the moveable component of the charging mechanism comprises the rotatable disc 124. At least a portion of the rotatable disc 124 is exposed on an exterior of the fluid charger 104 so as to enable a user to rotate the rotatable disc 124 about the rotational axis 176 thereof between the first position and the second position. Rotation of the rotatable disc 124 between the first position and the second position causes translational movement of the base member, here the piston chamber plate 136, in a direction parallel to the rotational axis 176 of the rotatable disc 124 (FIG. 4) and parallel to the longitudinal axes 211 of the pistons 168 and respective piston chambers 170 (FIGS. 3A and 3B).

The rotatable disc 124 is configured to enable simultaneous relative movement, in unison, between the pistons 168 and piston chambers 170. The cooperative, or in-unison, movement facilitates simultaneously changing volumes of the respective fluid pressure chambers 162 defined between the pistons 168 and piston chambers 170.

Referring again to FIG. 1, the rotatable disc 124 may include a marker 201 at a fixed location on the rotatable disc 124. The upper housing 122 also may include a first marker 202 and a second marker 204 at fixed locations thereon. The rotatable disc 124 may be rotatable relative to the upper housing 122 between a first rotational position in which the marker 201 on the rotatable disc 124 is aligned with the first marker 202 on the upper housing 122, and a second rotational position in which the marker 201 on the rotatable disc 124 is aligned with the second marker 204 on the upper housing 122.

In this configuration, rotating the rotatable disc 124 from the first rotational position to the second rotational position will resulting in charging of the lumens 106 and pressure transmission chambers 118 of the catheter 102 with fluid, while rotating the rotatable disc 124 from the second rotational position to the first rotational position will result in withdrawing fluid from the lumens 106 and pressure transmission chambers 118 of the catheter 102 (i.e., deflation of the pressure transmission chambers 118).

The fluid charger 104 may be configured such that, as the charging mechanism is actuated in the manner causing fluid to be withdrawn from the lumens 106 and pressure transmission chambers 118 of the catheter 102 (e.g., rotation of the rotatable disc 124 from the second rotational position to the first rotational position in the embodiment shown in the figures), a passageway is opened between each pressure chamber 162 and the ambient environment outside the fluid charger 104.

For example, as shown in FIG. 3B, a channel 173 may be formed through a sidewall of the piston chamber plate 136 defining each piston chamber 170 proximate the distal end thereof. In this configuration, as the piston 168 is withdrawn from within the respective piston chamber 170, once the end of the piston 168 passes the level of the channel 173, fluid communication is provided between the external ambient environment and the pressure chamber 162 through the respective channel 173 and the lumens 106 and associated pressure transmission chambers 118 assume a fully un-charged state open to atmosphere. As the piston 168 is moved in the opposite direction into the piston chamber 170, once the end of the piston 168 moves past the channel 173, the piston 168 seals off the channel 173, fluid communication between the external ambient environment and the pressure chamber 162 is interrupted, and further movement displaces fluid in the pressure chamber 162 into the fluid conduits 166 and into the lumens 106 and pressure transmission chambers 118 within the catheter 102.

Although the moveable component is depicted as a rotatable disc 124 in the figures, the moveable component could be any other moveable component or device, such as, for example, a knob with gears converting rotation of the knob to translational movement of the piston chamber plate 136 axes 211, or a sliding lever with a rack and pinion that converts sliding movement of the lever to translational movement of the piston chamber plate 136 axes 211.

Referring to FIGS. 3A and 3B, a threaded recess 172 may be formed at a central location in the piston plate 134, and an axially aligned unthreaded bore 174 may be formed through the piston chamber plate 136. A threaded bolt (not shown) with threads complementary to the threads of the threaded recess 172 may be passed through the unthreaded bore 174 of the piston chamber plate 136 and secured to the threaded recess 172. In this configuration, the piston chamber plate 136 is secured to the piston plate 134 by the threaded bolt in a manner that allows the piston chamber plate 136 to slide along the bolt relative to the piston plate 134 in the direction parallel to the axes 211 (FIG. 3A) and the rotational axis 176 (FIG. 4).

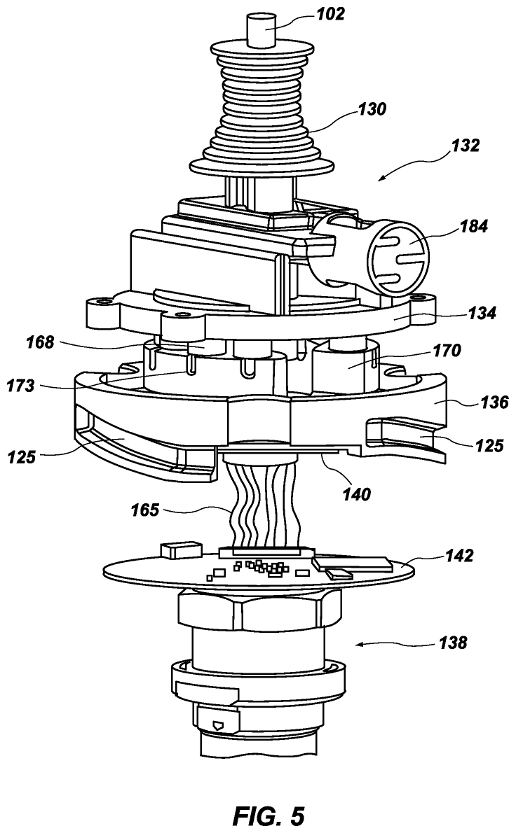

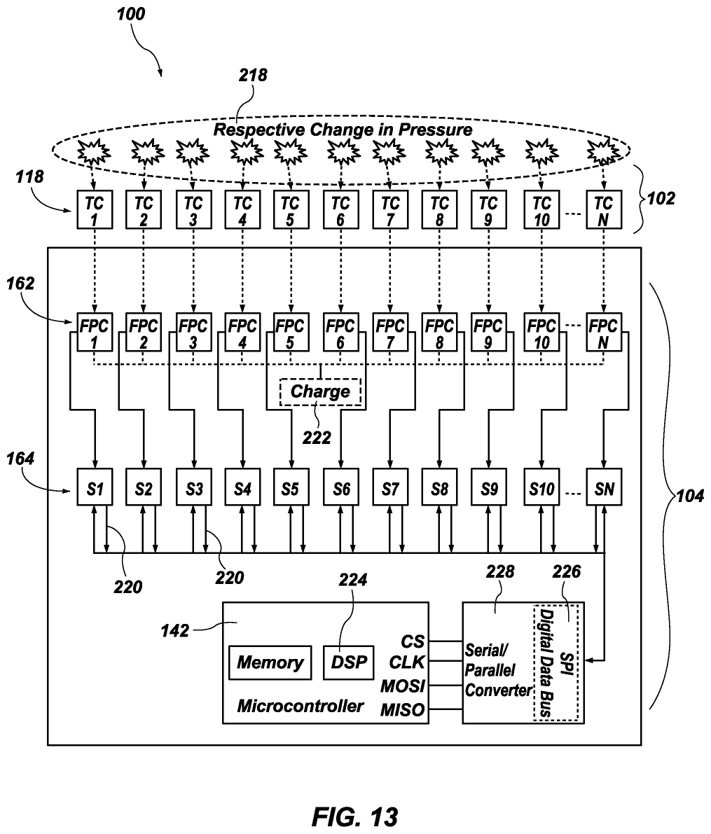

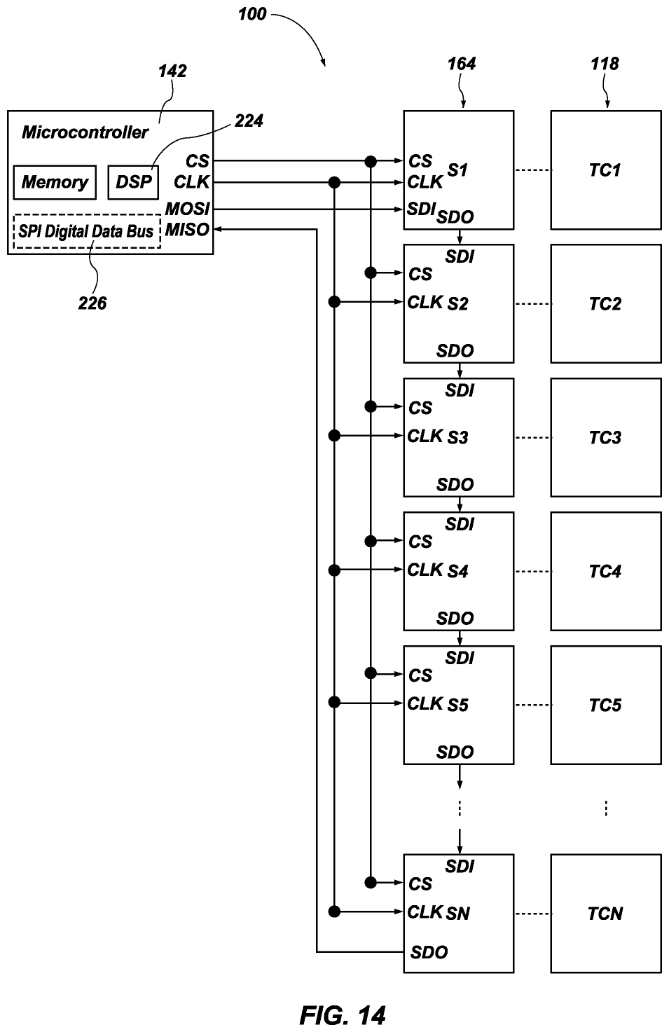

FIG. 5 illustrates the push button release mechanism 132 that is used to releasably couple with the catheter connector 130. The piston plate 134, piston chamber plate 136, and sensor PCB 140 are also illustrated in FIG. 5. The electrical connector 138 is also shown in FIG. 5. As shown therein, a microcontroller PCB 142 may be attached to an end of the electrical connector 138. One or more threaded nuts may lock the electrical connector 138 to the lower housing 128. The microcontroller PCB 142 may include one or more of signal processors, memory devices, and other electronic components used to acquire and process the electrical signals received from the pressure sensors 164 and sensor PCB 140. A flexible cable 165 (e.g., a ribbon cable) may be used to provide electrical connections between the sensor PCB 140 and the microcontroller PCB 142 to enable maintenance of electrical connections between the sensor PCB 140 and the microcontroller PCB 142 while allowing relative movement between the sensor PCB 140 and the microcontroller PCB 142. Power rails formed on and/or in the sensor PCB 140 may be shared among each of the plurality of pressure sensors 164, further facilitating the shared power bus features described in further detail below with reference to FIGS. 13 and 14.

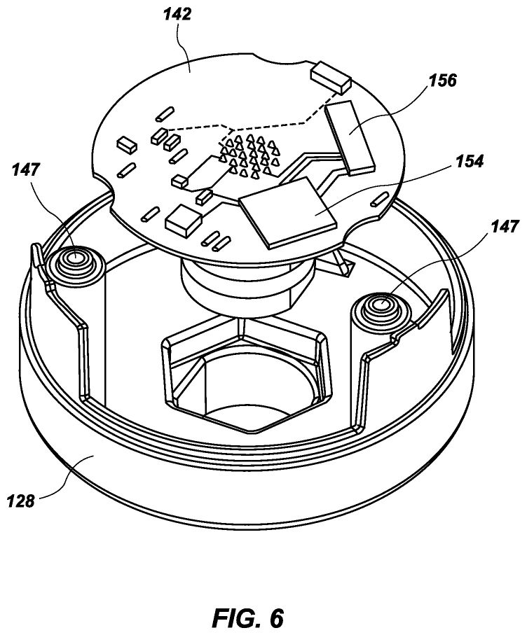

FIG. 6 illustrates the lower housing 128 and the microcontroller PCB 142. The lower housing 128 includes recesses for receiving fasteners (e.g., bolts or screws) used to secure the lower housing 128 to the piston plate 134 and upper housing 122. The microcontroller PCB 142 includes, among other things, a microcontroller 154 and a PCB connector 156 (e.g., for attaching the microcontroller PCB 142 via the flexible cable 165 to the sensor PCB 140). The microcontroller 154 may be tasked with encoding the data received from the pressure sensors 164 to prevent unintended access to the data, and to relay the data to an external control unit. The microcontroller PCB 142 of the lower housing 128 may be rigidly attached to the lower housing 128. Although not shown in FIG. 6, fasteners such as bolts or screws may be used to secure the microcontroller PCB 142 to the lower housing 128. In some embodiments, the lower housing 128 may include fastener mounts 147 that are complementarily sized and located relative to fastener openings on the piston plate 134, such that the plurality of pistons 168 (FIGS. 3A and 3B) are rigidly connected to the lower housing 128, in addition to the upper housing 122.

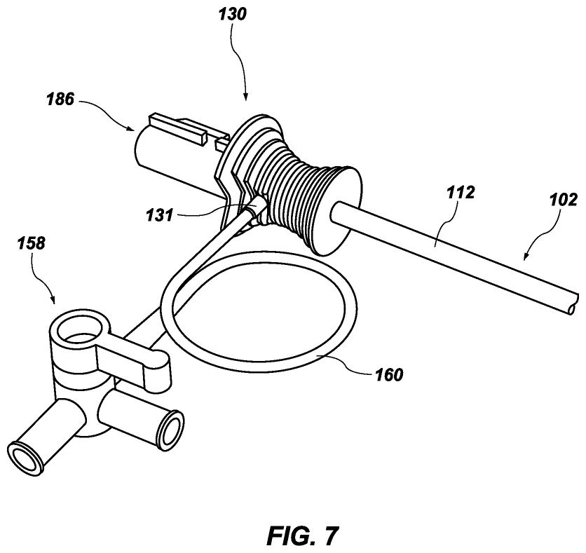

FIG. 7 illustrates the catheter connector 130 coupled to the proximal end 112 of the catheter 102, but detached from the fluid charger 104. As shown therein, the catheter connector 130 includes a coupling member 131 sized and configured to couple with, for example, tubing 160, on the exterior of the catheter connector 130. An axis of the coupling member 131 may be oriented at an angle (e.g., about a 90° angle) relative to an axis of the catheter connector 130. The coupling member 131 is in fluid communication with the additional lumen 114 and the inflatable balloon 120 at the distal end 116 of the catheter 102. A fluid valve 158 (e.g., a stop cock) may be provided along the tubing 160 as necessary or desired, and a source of pressurized fluid (e.g., gas or air) may be coupled to the fluid valve 158 and used to inflate the balloon 120 as needed or desirable during operation of the manometry device 100.

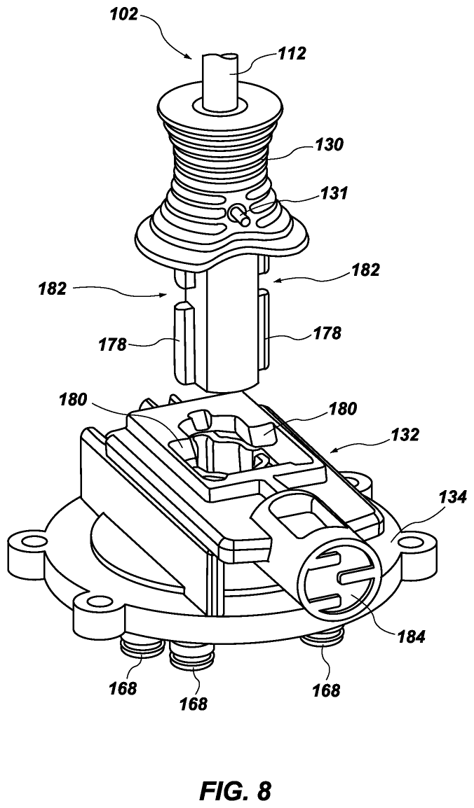

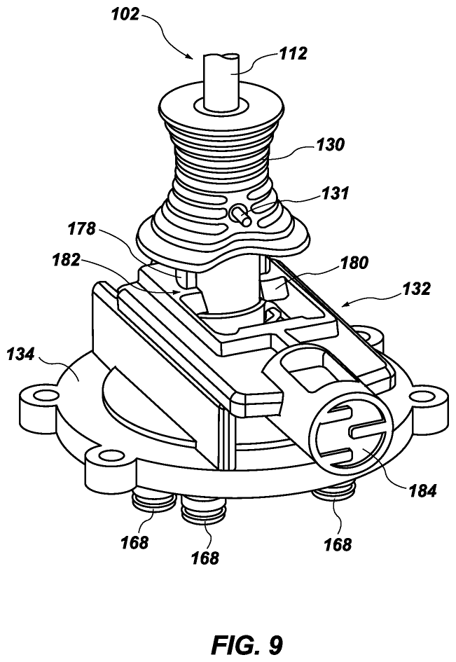

FIG. 8 illustrates the catheter connector 130 aligned with, but separated from, the corresponding push button release mechanism 132, which is fixedly attached to the piston plate 134 in this embodiment. FIG. 9 illustrates the catheter connector 130 of FIG. 8 operationally coupled with the release mechanism 132. Referring collectively to FIGS. 8 and 9, the catheter connector 130 and the corresponding push button release mechanism 132 may be complementarily sized and configured so as to ensure proper alignment and a fluid tight connection between the fluid charger 104 and the catheter 102. In other words, the catheter connector 130 is geometrically configured to connect with, or be seated within, the push button release mechanism 132. For example, an opening of the push button release mechanism 132 may be similar in shape, dimension, and alignment relative to the proximal end 186 of the catheter connector 130. The shape of the opening in the push button release mechanism 132 may be restrictive, such that only a single and specific relative alignment and orientation of the catheter connector 130 relative to the release mechanism enables the catheter connector 130 to be inserted into and coupled with the release mechanism 132. The push button release mechanism 132 may be rigidly coupled to the piston plate 134, as previously mentioned.

Vertical flanges 178 may be provided on the catheter connector 130 and positioned above complementary sloped surfaces 180 of the push button release mechanism 132. The vertical flanges 178 may include notches 182 with which features of the release mechanism 132 may mechanically interlock so as to lock the catheter connector 130 in place within the release mechanism 132 during operation. Manual pushing of the button 184 in the direction transverse to the axis of the catheter 102, against a spring biasing mechanism of the push button release mechanism 132, may cause the feature of the release mechanism 132 to withdraw from the notches 182 in the vertical flanges 178, thereby allowing the catheter connector 130 to be withdrawn from the release mechanism 132 while pressing the button 184.

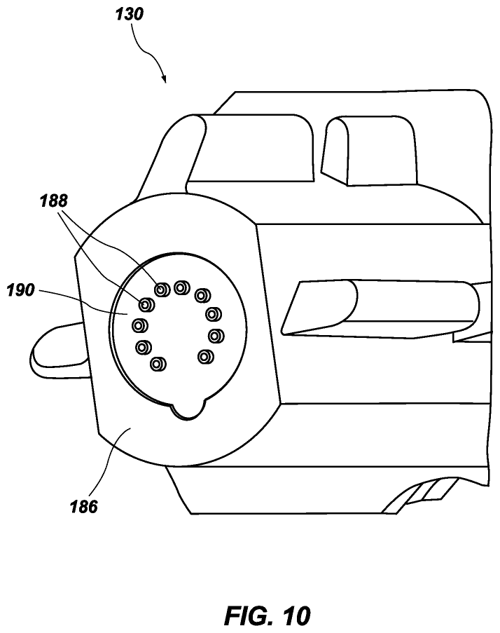

FIG. 10 illustrates a proximal end 186 of the catheter connector 130. As shown therein, rigid tubes 188 may be provided within the catheter connector 130, and each of these rigid tubes 188 may extend through the catheter connector 130 and into a respective one lumen 106 of the lumens 106 within the catheter 102. An elastomeric gasket 190 may be provided at this proximal end 186 of the catheter connector 130 to assist in providing a fluid tight seal around each rigid tube 188 at the interface between the catheter connector 130 and the fluid charger 104.

As previously mentioned, the catheter 102 may be a single-use disposable catheter, and the catheter connector 130 may be a permanent part of that disposable catheter not intended for reuse. Thus, the catheter connector 130 may be permanently and securely fastened to the catheter 102 by the manufacturer upon manufacture thereof. For example, the proximal end 112 of the catheter 102 may be secured with an adhesive within the catheter connector 130, In other embodiments, the catheter connector 130 may be formed around the rigid tubes 188 and/or the proximal end 112 of the catheter 102 by, for example, over-molding or by additive manufacturing (e.g., 3D printing).

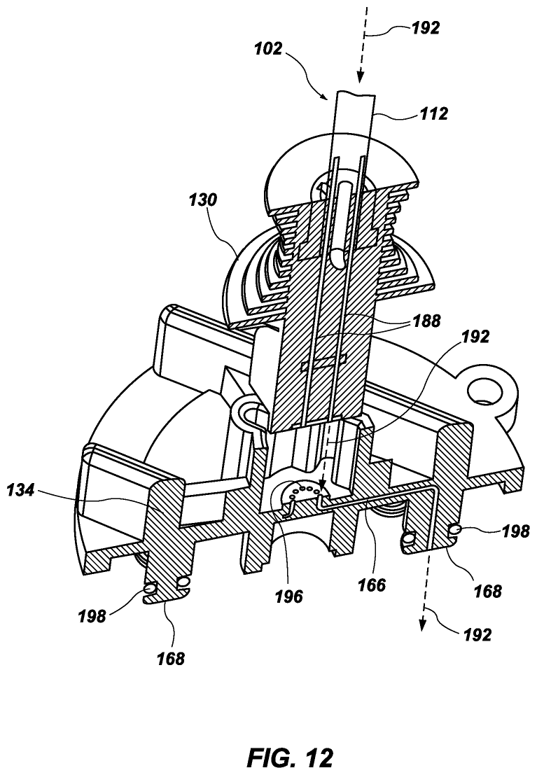

FIG. 12 is a longitudinal cross-sectional view of the proximal end 112 of the catheter 102, the catheter connector 130, and the piston plate 134. The catheter connector 130 is shown retracted a distance from the operational position relative to the piston plate 134. The release mechanism 132 and other components of the fluid charger 104 are not illustrated to clearly show the respective fluid pathways between the lumens 106 of the catheter 102, through the catheter connector 130, and through the fluid conduits within the piston plate 134 to the pressure chambers 162 (FIGS. 3A and 3B).

Referring to FIG. 12, the ends of the rigid tubes 188 are inserted into corresponding holes 196 formed in the piston plate 134 upon insertion of the catheter connector 130 into the release mechanism 132, as indicated by the directional arrow 192 in FIG. 12. Upon complete insertion of the catheter connector 130 into the release mechanism 132, and in use of the device 100, the gasket 190 is compressed between the catheter connector 130 and the piston plate 134 around the ends of the rigid tubes 188 to form a fluid-tight seal therebetween. Each piston 168 may carry an O-ring 198 to provide a fluid-tight seal between the piston 168 and the respective piston chamber 170 in the fluid pressure chamber 162.

FIG. 13 is a is a schematic diagram of a diagnostic manometry device 100 in accordance with embodiments of the present disclosure, including one such as that illustrated in FIG. 1. As shown therein, the catheter 102 includes a plurality of pressure transmission chambers 118 (e.g., TC₁, TC₂, . . . TC_{N}). The plurality of pressure transmission chambers 118 (e.g., TC₁, TC₂, . . . TC_{N}) are each in fluid communication with a respective fluid pressure chamber 162 in the fluid charger 104 (e.g., FPC₁, FPC₂, . . . FPC_{N}). Each pressure sensor (e.g., S₁, S₂, . . . S_{N}) of a plurality of pressure sensors 164 of the fluid charger 104 is configured for measuring fluid pressure and/or fluctuations in fluid pressure in a respective fluid pressure chamber 162. Each pressure sensor 164 (e.g., S₁, S₂, . . . S_{N}) may comprise a digital pressure sensor configured to provide one or more digital output signals 220 indicative of a pressure detected by the respective pressure sensor 164 (e.g., S₁, S₂, . . . S_{N}) of the plurality or pressure sensors 164. Initially, the plurality of fluid pressure chambers 162 may be simultaneously charged 222, in unison, with fluid as previously described herein to establish a baseline pressure and provide a calibration point for the plurality or pressure sensors 164, thereby enabling the detection of a change in fluid pressure within the pressure transmission chambers 118 caused by a change in pressure 218 outside and adjacent each respective pressure transmission chamber 118. The respective fluid pressure changes are detected by the respective pressure sensors 164, and digital output signals 220 indicative of the pressure changes may be transmitted to a digital signal processor (DSP) 224.

The digital signal processor (DSP) 224 receives the one or more digital output signals 220 from one or all pressure sensors of the plurality of pressure sensors 164 through a serial peripheral interface (SPI) digital data bus 226. The SPI digital data bus 226 is positioned and electrically connected between the DSP 224 and the plurality of pressure sensors 164. The SPI digital data bus 226 may include, for example, four associated signal lines, including a clock line (CLK), a chip select line (CS) or an inverted chip select line (CS_N—not shown), a master input slave output (MISO) line, and a master output slave input (MOSI) line. Corresponding contact pads, including contact pads for ground (GND) and power (PWR), may be found on a chip set for the microcontroller 154. It is noted that power may be direct current (e.g., from a battery, such as a lithium ion battery), or may be alternating current (e.g., from the electrical connector 138). The DSP 224 may be configured to sequentially connect sample the signal lines corresponding to each pressure sensor 164. Each sampling may require, as a non-limiting example, several microseconds. Thus, the DSP 224 can acquire and process the data from each pressure sensor 164 several times a second. Due to the rapid rate of data sampling, and the very short required sampling time, the digital output signal from each of the pressure sensors 164 may be continuously and constantly monitored from the perspective of the user and patient.

By using digital sensors that output digital signals in conjunction with a DSP 224 and SPI digital data bus 226, the number of signal lines can maintained at four or less, for example, regardless of the number of pressure sensors 164 and corresponding pressure chambers 162 in the fluid charger 104 (and lumens 106 and pressure transmission chambers 118 in the catheter 102), which simplifies construction and improves scalability relative to previously known devices that employ analog pressure sensors. Furthermore, the use of a plurality of pressure sensors 164 that provide one or more digital output signals 220 eliminates the need for analog to digital conversion, saving space within the diagnostic manometry device 100 and reducing components included on PCBs. The use of a DSP 224 and SPI digital data bus 226 reduces wiring and interconnects, which reduces manufacturing costs and provides opportunities for signal transfer with decreased interference relative to conventional diagnostic manometry devices.

FIG. 14 is schematic diagram of a diagnostic manometry device 100 in accordance with additional embodiments of the present disclosure, including one such as that illustrated in FIG. 1. Referring to FIG. 14, in alternative embodiments, the SPI serial to parallel converter 228 may be removed, so as to reduce costs. In these embodiments, the SPI nodes (e.g., plurality of pressure sensors 164) may be daisy chained together, receiving and relaying data as incremented values to the SPI digital data bus 226 of the microcontroller 154.

Additional embodiments of the present disclosure include methods of using a manometry device 100 as described herein to perform a diagnostic manometry procedure. In according with such methods, the distal end 116 of the catheter 102 (including the inflatable balloon 120 and at least a portion of the pressure transmission chambers 118) may be inserted into the body of a patient to facilitate measurement of pressure and/or fluctuations in pressure outside and adjacent the pressure transmission chambers 118. In use, changes in pressure outside the catheter adjacent the pressure transmission chambers 118 are transmitted through the flexible walls of the catheter body to the fluid within the pressure transmission chambers 118, and hence to the fluid within the fluid pressure chambers 162 within the fluid charger 104. Thus, detected changes in fluid pressure, such as air pressure, within the pressure chambers 162 as detected by the plurality of pressure sensors 164 correlate with changing pressures within the body of the patient outside the catheter adjacent the pressure transmission chambers 118.

The catheter 102 may be coupled to the fluid charger 104 before or after insertion of the catheter 102 into the body of the patient. If the catheter 102 is coupled to the fluid charger 104 before insertion of the catheter 102 into the body of the patient, the charging mechanism may be manipulated so as to ensure that fluid is withdrawn from the pressure transmission chambers 118 of the catheter prior to insertion of the catheter 102 into the body of the patient for ease of insertion and minimization of discomfort to the patient. To couple the catheter 102 to the fluid charger 104, the catheter connector 130 is inserted into the translationally-biased push button release mechanism 132 as previously described with reference to FIGS. 8 and 9. Bottom surfaces of the vertical flanges 178 engage sloped surfaces 180 of the push button release mechanism 132, thereby translating the push button release mechanism 132 until the biasing force laterally translates the sloped surfaces 180 into notches 182 (see FIG. 8).

The pressure transmission chambers 118 may be charged with fluid using the fluid charger 104 as previously described herein. In particular, the charging mechanism of the fluid charger 104 may be manipulated so as to simultaneously move fluid from the fluid pressure chambers 162 into the respective lumens 106 and into the pressure transmission chambers 118, reducing the risk of procedural error.

Any required calibration of the pressure sensors 164 then may be performed, after which manometry diagnostic testing may be performed in accordance with generally known techniques. For example, in anorectal manometry techniques, the patient may be asked to “squeeze” their rectum to simulate the physiological action of hindering a bowel movement, followed by asking the patient to “push” so as to simulate the physiological action of attempting to initiate a bowel movement. The diagnostic manometry device 100 can be used to detect whether the patients anus and rectum are responding as the patient intends, so as to identify any neurological or muscular dysfunction. Detected pressure relative to the diagnostic manometry device 100 may be proportional to pressures generated by the patient. The inflatable balloon 120 may also be positioned in the colon of the patient and inflated by forcing fluid into the additional lumen 114 through the coupling member 131 on the catheter connector 130. This may simulate the accumulation of feces in the colon, which would be expected to cause the patient to feel the urge to defecate. In a healthy patient, this would elicit a contraction of the colon and rectum of the patient, which would be detectable using the diagnostic manometry device 100. If the patient has a relevant neurological or muscular dysfunction (e.g., disorder or injury), the detected pressure changes (or lack thereof) would not match the expected pressure changes outside the catheter adjacent the pressure transmission chambers 118.

After the procedure, the catheter 102 may be withdrawn from the patient, and the catheter connector 130 can be removed from the fluid charger 104 by applying translational force to the push button 184 and extracting the catheter connector 130 fluid the release mechanism, after which the catheter 102 and catheter connector 130 may be discarded. The fluid charger 104 then may be cleaned and sterilized as needed for reuse.

Additional non-limiting example embodiments of the present disclosure are set forth herein below:

Embodiment 1: A diagnostic manometry device, comprising: a catheter including a plurality of lumens; and a fluid charger configured to operatively couple with the catheter, the fluid charger including: a plurality of fluid pressure chambers, each fluid pressure chamber of the plurality of fluid pressure chambers configured to be in fluid communication with a respective lumen of the plurality of lumens when the catheter is operatively coupled with the fluid charger; a plurality of pressure sensors, each pressure sensor of the plurality of pressure sensors located and configured to measure a fluid pressure change in a respective fluid pressure chamber of the plurality of fluid pressure chambers; and a charging mechanism for moving fluid simultaneously from the fluid pressure chambers of the plurality of fluid pressure chambers into the respective lumens of the plurality of lumens when the catheter is operatively coupled with the fluid charger.

Embodiment 2: The diagnostic manometry device of Embodiment 1, wherein the plurality of lumens comprises at least four lumens.

Embodiment 3: The diagnostic manometry device of Embodiment 2, wherein the plurality of lumens comprises at least ten lumens.

Embodiment 4: The diagnostic manometry device of any one of Embodiments 1 through 3, wherein the catheter comprises a plurality of pressure transmission chambers, each pressure transmission chamber of the plurality of pressure transmission chambers in fluid communication with a respective lumen of the plurality of lumens.

Embodiment 5: The diagnostic manometry device of Embodiment 4, wherein each pressure transmission chamber comprises an expandable cylindrical balloon carried concentrically along the catheter.

Embodiment 6: The diagnostic manometry device of any one of Embodiments 1 through 5, wherein the catheter comprises an additional lumen coupled with an inflatable balloon, the additional lumen not configured to be in fluid communication with a fluid pressure chamber of the plurality of fluid pressure chambers of the fluid charger.

Embodiment 7: The diagnostic manometry device of any one of Embodiments 1 through 6, wherein the fluid charger further comprises a plurality of pistons and a plurality of piston chambers, each piston chamber sized and configured to receive a respective piston of the plurality of pistons therein in a fluid-tight manner, each piston and respective piston chamber defining a respective fluid pressure chamber of the plurality of fluid pressure chambers therebetween.

Embodiment 8: The diagnostic manometry device of Embodiment 7, wherein the charging mechanism is configured to enable relative movement between the plurality of pistons and the plurality of piston chambers in unison so as to simultaneously change volumes of the fluid pressure chambers of the plurality of fluid pressure chambers defined between the respective pistons and piston chambers.

Embodiment 9: The diagnostic manometry device of Embodiment 8, wherein the charging mechanism comprises a moveable component moveable between a first position and a second position, movement of the moveable component from the first position to the second position causing the relative movement between the plurality of pistons and the plurality of piston chambers.

Embodiment 10: The diagnostic manometry device of Embodiment 9, wherein the charging mechanism further comprises a base member, one of the plurality of pistons and the plurality of piston chambers being rigidly connected to the base member.

Embodiment 11: The diagnostic manometry device of Embodiment 10, wherein the moveable component of the charging mechanism is operatively coupled with the base member of the charging mechanism such that movement of the moveable component from the first position to the second position causes the base member to move in a direction parallel to longitudinal axes of the pistons and respective piston chambers.

Embodiment 12: The diagnostic manometry device of Embodiment 11, wherein the moveable component of the charging mechanism comprises a rotatable disc, at least a portion of the rotatable disc being exposed on an exterior of the fluid charger so as to enable a user to rotate the rotatable disc about a rotational axis thereof between the first position and the second position, rotation of the rotatable disc between the first position and the second position causing translational movement of the base member in a direction parallel to the rotational axis of the rotatable disc and parallel to the longitudinal axes of the pistons and respective piston chambers.

Embodiment 13: The diagnostic manometry device of any one of Embodiments 1 through 12, wherein each pressure sensor of the plurality of pressure chambers comprises a digital pressure sensor configured to provide a digital output signal indicative of a pressure detected by the pressure sensor.

Embodiment 14: The diagnostic manometry device of Embodiment 13, wherein the fluid charger further comprises a digital signal processor configured to receive the digital output signal from each pressure sensor of the plurality of pressure sensors through a serial peripheral interface bus between the digital signal processor and the plurality of pressure sensors.

Embodiment 15: A fluid charger for use with a diagnostic manometry device, comprising: a housing; a catheter connector configured to couple with a proximal end of a catheter including lumens defined within a body of the catheter; fluid pressure chambers, each fluid pressure chamber in fluid communication with a respective fluid conduit leading to the catheter connector so as to fluidly couple each fluid pressure chamber with a respective lumen of a catheter when the catheter is operatively connected to the fluid charger; pressure sensors for measuring fluid pressure changes in the fluid pressure chambers; and a charging mechanism for moving fluid simultaneously from the fluid pressure chambers through the fluid conduits toward the catheter connector.

Embodiment 16: The fluid charger of Embodiment 15, wherein the fluid pressure chambers comprise at least four fluid pressure chambers.

Embodiment 17: The fluid charger of Embodiment 16, wherein the fluid pressure chambers comprise at least ten fluid pressure chambers.

Embodiment 18: The fluid charger of any one of Embodiments 15 through 17, further comprising a fluid connector in fluid communication with another fluid conduit leading to the catheter connector so as to couple with another lumen of a catheter when the catheter is operatively connected to the fluid charger, the fluid connector and another fluid conduit not in fluid communication with any fluid pressure chamber of the fluid charger.

Embodiment 19: The fluid charger of any one of Embodiments 15 through 18, further comprising pistons and piston chambers, each piston chamber sized and configured to receive a respective piston therein in a fluid-tight manner, each piston and respective piston chamber defining a respective one of the fluid pressure chambers within the respective piston chamber adjacent the piston.

Embodiment 20: The fluid charger of Embodiment 19, wherein the charging mechanism is configured to enable relative movement between the pistons and the piston chambers in unison so as to simultaneously change volumes of the fluid pressure chambers.

Embodiment 21: The fluid charger of Embodiment 20, wherein the charging mechanism comprises a moveable component moveable between a first position and a second position, movement of the moveable component from the first position to the second position causing the relative movement between the pistons and piston chambers.

Embodiment 22: The fluid charger of Embodiment 21, wherein the charging mechanism further comprises a base member, the pistons or the piston chambers being rigidly connected to the base member.

Embodiment 23: The fluid charger of Embodiment 22, wherein the moveable component of the charging mechanism is operatively coupled with the base member of the charging mechanism such that movement of the moveable component from the first position to the second position causes the base member to move in a direction parallel to longitudinal axes of the pistons and respective piston chambers.

Embodiment 24: The fluid charger of Embodiment 23, wherein the moveable component of the charging mechanism comprises a rotatable disc, at least a portion of the rotatable disc being exposed on an exterior of the fluid charger so as to enable a user to rotate the rotatable disc about a rotational axis thereof between the first position and the second position, rotation of the rotatable disc between the first position and the second position causing translational movement of the base member in a direction parallel to the rotational axis of the rotatable disc and parallel to the longitudinal axes of the pistons and respective piston chambers.

Embodiment 25: The fluid charger of any one of Embodiments 15 through 24, wherein each of the pressure sensors comprises a digital pressure sensor configured to provide a digital output signal indicative of a pressure detected by the pressure sensor.

Embodiment 26: The fluid charger of Embodiment 25, further comprising a digital signal processor configured to receive the digital output signal from each of the pressure sensors through a serial peripheral interface bus.

Embodiment 27: A method of performing a diagnostic manometry procedure on a patient, comprising: coupling a catheter including a plurality of lumens to a fluid charger, the fluid charger including: a plurality of fluid pressure chambers, each fluid pressure chamber of the plurality of fluid pressure chambers in fluid communication with a respective lumen of the plurality of lumens upon the coupling of the catheter to the fluid charger; a plurality of pressure sensors, each pressure sensor of the plurality of pressure sensors located and configured to measure a fluid pressure change in a respective fluid pressure chamber of the plurality of fluid pressure chambers; and a charging mechanism for moving fluid simultaneously from the fluid pressure chambers of the plurality of fluid pressure chambers into the respective lumens of the plurality of lumens; inserting the catheter into a body of patient; manipulating the charging mechanism so as to simultaneously move fluid from the fluid pressure chambers of the plurality of fluid pressure chambers into the respective lumens of the plurality of lumens; and detecting changes in pressure in the fluid pressure chambers of the plurality of fluid pressure chambers using the pressure sensors of the plurality of pressure sensors.

Embodiment 28: The method of Embodiment 27, wherein the fluid is a gas.

Embodiment 29: The method of Embodiment 27 or 28, further comprising using a diagnostic manometry device in accordance with any one of Embodiments 1 through 14 to perform the diagnostic manometry procedure on a patient.

The embodiments of the disclosure described above and illustrated in the accompanying drawings do not limit the scope of the disclosure. Any equivalent embodiments are within the scope of this disclosure. Indeed, various modifications of the disclosure, in addition to those shown and described herein, such as alternate useful combinations of the elements described, will become apparent to those skilled in the art from the description. Such modifications and embodiments may also fall within the scope of the invention as defined by the appended claims and equivalents.

What is claimed is:

1. A diagnostic manometry device, comprising:
a catheter including a plurality of lumens; and
a fluid charger configured to operatively couple with the catheter, the fluid charger including:
a plurality of fluid pressure chambers, each fluid pressure chamber of the plurality of fluid pressure chambers configured to be in fluid communication with a respective lumen of the plurality of lumens when the catheter is operatively coupled with the fluid charger;
a plurality of pressure sensors, each pressure sensor of the plurality of pressure sensors located and configured to measure a fluid pressure change in a respective fluid pressure chamber of the plurality of fluid pressure chambers; and
a charging mechanism for moving fluid simultaneously from the fluid pressure chambers of the plurality of fluid pressure chambers into the respective lumens of the plurality of lumens when the catheter is operatively coupled with the fluid charger.

2. The diagnostic manometry device of claim 1, wherein the plurality of lumens comprises at least four lumens.

3. The diagnostic manometry device of claim 2, wherein the plurality of lumens comprises at least ten lumens.

4. The diagnostic manometry device of claim 1, wherein the catheter comprises a plurality of pressure transmission chambers, each pressure transmission chamber of the plurality of pressure transmission chambers in fluid communication with a respective lumen of the plurality of lumens.

5. The diagnostic manometry device of claim 4, wherein each pressure transmission chamber comprises an expandable cylindrical balloon carried concentrically along the catheter.

6. The diagnostic manometry device of claim 1, wherein the catheter comprises an additional lumen fluidly coupled with an inflatable balloon, the additional lumen not configured to be in fluid communication with a fluid pressure chamber of the plurality of fluid pressure chambers of the fluid charger.

7. The diagnostic manometry device of claim 1, wherein the fluid charger further comprises a plurality of pistons and a plurality of piston chambers, each piston chamber sized and configured to receive a respective piston of the plurality therein in a fluid-tight manner, each piston and respective piston chamber defining a respective fluid pressure chamber of the plurality therebetween.

8. The diagnostic manometry device of claim 7, wherein the charging mechanism is configured to enable relative movement between the plurality of pistons and the plurality of piston chambers in unison so as to simultaneously change volumes of the fluid pressure chambers of the plurality defined between the respective pistons and piston chambers.

9. The diagnostic manometry device of claim 8, wherein the charging mechanism comprises a moveable component moveable between a first position and a second position, movement of the moveable component from the first position to the second position causing the relative movement between the plurality of pistons and the plurality of piston chambers.

10. The diagnostic manometry device of claim 9, wherein the charging mechanism further comprises a base member, one of the plurality of pistons and the plurality of piston chambers being rigidly connected to the base member.

11. The diagnostic manometry device of claim 10, wherein the moveable component of the charging mechanism is operatively coupled with the base member of the charging mechanism such that movement of the moveable component from the first position to the second position causes the base member to move in a direction parallel to longitudinal axes of the pistons and respective piston chambers.

12. The diagnostic manometry device of claim 11, wherein the moveable component of the charging mechanism comprises a rotatable disc, at least a portion of the rotatable disc being exposed on an exterior of the fluid charger so as to enable a user to rotate the rotatable disc about a rotational axis thereof between the first position and the second position, rotation of the rotatable disc between the first position and the second position causing translational movement of the base member in a direction parallel to the rotational axis of the rotatable disc and parallel to the longitudinal axes of the pistons and respective piston chambers.

13. The diagnostic manometry device of claim 1, wherein each pressure sensor of the plurality of pressure sensors comprises a digital pressure sensor configured to provide a digital output signal indicative of a pressure detected by the pressure sensor.

14. The diagnostic manometry device of claim 13, wherein the fluid charger further comprises a digital signal processor configured to receive the digital output signal from each pressure sensor of the plurality of pressure sensors through a serial peripheral interface bus between the digital signal processor and the plurality of pressure sensors.

15. A fluid charger for use with a diagnostic manometry device, comprising:
a housing;
a catheter connection mechanism configured to couple with a proximal end of a catheter including lumens defined within a body of the catheter;
fluid pressure chambers, each fluid pressure chamber in fluid communication with a respective fluid conduit leading to the catheter connection mechanism so as to fluidly couple each fluid pressure chamber with a respective lumen of a catheter when the catheter is operatively connected to the fluid charger;
pressure sensors for measuring fluid pressure changes in the fluid pressure chambers; and
a charging mechanism for moving fluid simultaneously from the fluid pressure chambers through the fluid conduits toward the catheter connector.

16. The fluid charger of claim 15, wherein the fluid pressure chambers comprise at least four fluid pressure chambers.

17. The fluid charger of claim 15, wherein the fluid pressure chambers comprise at least ten fluid pressure chambers.

18. The fluid charger of claim 15, further comprising a fluid connector in fluid communication with another fluid conduit leading to the catheter connection mechanism so as to couple with another lumen of a catheter when the catheter is operatively connected to the fluid charger, the fluid connector and another fluid conduit not in fluid communication with any fluid pressure chamber of the fluid charger.

19. The fluid charger of claim 15, further comprising pistons and piston chambers, each piston chamber sized and configured to receive a respective piston therein in a fluid-tight manner, each piston and respective piston chamber defining a respective one of the fluid pressure chambers within the respective piston chamber adjacent the piston.

20. The fluid charger of claim 19, wherein the charging mechanism is configured to enable relative movement between the pistons and the piston chambers in unison so as to simultaneously change volumes of the fluid pressure chambers.

21. The fluid charger of claim 20, wherein the charging mechanism comprises a moveable component moveable between a first position and a second position, movement of the moveable component from the first position to the second position causing the relative movement between the pistons and piston chambers.

22. The fluid charger of claim 21, wherein the charging mechanism further comprises a base member, the pistons or the piston chambers being rigidly connected to the base member.

23. The fluid charger of claim 22, wherein the moveable component of the charging mechanism is operatively coupled with the base member of the charging mechanism such that movement of the moveable component from the first position to the second position causes the base member to move in a direction parallel to longitudinal axes of the pistons and respective piston chambers.

24. The fluid charger of claim 23, wherein the moveable component of the charging mechanism comprises a rotatable disc, at least a portion of the rotatable disc being exposed on an exterior of the fluid charger so as to enable a user to rotate the rotatable disc about a rotational axis thereof between the first position and the second position, rotation of the rotatable disc between the first position and the second position causing translational movement of the base member in a direction parallel to the rotational axis of the rotatable disc and parallel to the longitudinal axes of the pistons and respective piston chambers.

25. The fluid charger of claim 15, wherein each of the pressure sensors comprises a digital pressure sensor configured to provide a digital output signal indicative of a pressure detected by the pressure sensor.

26. The fluid charger of claim 25, further comprising a digital signal processor configured to receive the digital output signal from each of the pressure sensors through a serial peripheral interface bus.

27. A method of performing a diagnostic manometry procedure on a patient, comprising:
coupling a catheter including a plurality of lumens to a fluid charger, the fluid charger including:
a plurality of fluid pressure chambers, each fluid pressure chamber of the plurality of fluid pressure chambers in fluid communication with a respective lumen of the plurality of lumens upon the coupling of the catheter to the fluid charger;
a plurality of pressure sensors, each pressure sensor of the plurality of pressure sensors located and configured to measure a fluid pressure change in a respective fluid pressure chamber of the plurality of fluid pressure chambers; and
a charging mechanism for moving fluid simultaneously from the fluid pressure chambers of the plurality of fluid pressure chambers into the respective lumens of the plurality of lumens;
inserting the catheter into a body of patient;
manipulating the charging mechanism so as to simultaneously move fluid from the fluid pressure chambers of the plurality of fluid pressure chambers into the respective lumens of the plurality of lumens; and
detecting changes in pressure in the fluid pressure chambers of the plurality of fluid pressure chambers using the pressure sensors of the plurality of pressure sensors.

28. The method of claim 27, wherein the fluid is a gas.
